(19) United States
(12) Patent Application Publication
(10) Pub. No.: US 2025/0304695 A1
(43) Pub. Date: Oct. 2, 2025

(54) Treatment for colorectal cancer using immune checkpoint inhibitors

(71) Applicant: Mirror Biologics, Inc., Wesley Chapel, FL (US)

(72) Inventor: Michael Har-Noy, Wesley Chapel, FL (US)

(21) Appl. No.: 19/092,659

(22) Filed: Mar. 27, 2025

Related U.S. Application Data
(60) Provisional application No. 63/571,265, filed on Mar. 28, 2024.
(60) Provisional application No. 63/631,043, filed on Apr. 8, 2024.
(60) Provisional application No. 63/702,989, filed on Oct. 3, 2024.
(60) Provisional application No. 63/733,785, filed on Dec. 13, 2024.

(51) Int. Cl. C07K 16/28; A61K 31/44; A61K 35/17; A61K 39/00; A61P 35/04

(52) U.S. Cl. CPC C07K 16/2818; A61K 31/44; A61K 35/17; A61P 35/04; C07K 16/2827; A61K 2039/507

(57) ABSTRACT

A method and composition is described of treating metastatic colorectal cancer in a patient, comprises administering activated allogeneic CD4+ T-cells to a patient; and administering at least one immune checkpoint inhibitor to the patient

### BACKGROUND

The present disclosure relates to a therapeutic composition and methods of use thereof for treating patients with immunologically cold, microsatellite stable (MSS), proficient mismatch repair (pMMR) metastatic solid tumors and metastatic solid tumors.

Immune Checkpoint Inhibitor (ICI) immunotherapy is most effective in immune effector cell infiltrated “hot” tumor lesions such lesions occur in deficient DNA mismatch repair/microsatellite instability high (dMMR/MSI-H) tumors such as colorectal cancer (CRC). However, most all metastatic CRC tumors are pMMR/MSS “cold” lesions, without significant immune cell infiltration, and are unresponsive to ICI.

### SUMMARY

A method of treating metastatic colorectal cancer in a patient, comprises administering activated allogeneic CD4+ T-cells to a patient; and administering at least one immune checkpoint inhibitor to the patient.

In a further embodiment the immune checkpoint inhibitor comprises any one of ipilimumab; pembrolizumab; nivolumab; atezolizumab; avelumab; durvalumab; cemiplimab; tremelimumab; retifanlimab; dostarlimab; toripalimab; or combinations thereof.

In a further embodiment the immune checkpoint inhibitor targets CTLA-4, PD-1, PD-L1, and/or LAG-3 checkpoint molecules.

In a further embodiment the method comprises administering regorafenib.

In a further embodiment the patient has a tumor.

In a further embodiment the method comprises reducing the size of the tumor or eliminating the tumor.

In a further embodiment the activated allogeneic CD4+ T-cells comprises an activated Th1 memory phenotype: CD4+, CD45RO+, CD62Llo, CD40Lhi, CD25+, IFN-γ+, and IL-4−.

In a further embodiment the method comprises wherein the activated allogeneic CD4+ T-cells are maintained in an activated state by continuous attachment to CD3/CD28-monoclonal antibody-coated microparticles.

In a further embodiment the administration comprises intradermal or intravenous and is administered more than once.

In a further embodiment the CD4+ T-cells are differentiated and expanded ex-vivo to become Th1-like effector cells prior to administration.

In a further embodiment the Th1-like effector cells are stored in liquid nitrogen as an intermediate prior to administration.

In a further embodiment the Th1-like effector cells after storage in the liquid nitrogen are thawed and activated with microbeads causing differentiation of the cells to an activated Th1 memory cell phenotype prior to administration.

In yet another embodiment the method comprises a method of treating metastatic colorectal cancer in a patient, comprising priming a patient comprising increasing a titer of circulating CD4+ Th1 memory cells in the patient by administering a therapeutic composition to the patient, wherein the patient has a tumor and the therapeutic composition comprises activated allogeneic CD4+ T-cells; and activating the circulating CD4+ Th1 memory cells in the patient; and administering an immune checkpoint inhibitor to the patient.

In a further embodiment the method comprises increasing the titer of circulating CD4+ Th1 memory cells in the patient is caused by administering an intradermal dose of the therapeutic composition to the patient.

In a further embodiment the method comprises activating the circulating CD4+ Th1 memory cells in the patient is caused by administering an intravenous dose of the therapeutic composition to the patient.

In a further embodiment the tumor comprises a “cold” tumor characterized by the tumor lacking tumor-infiltrating lymphocytes.

In a further embodiment the method comprises priming converts the “cold” tumor to a “hot” tumor, wherein the “hot” tumor comprises one or more of an increased level of tumor-infiltrating lymphocytes, an interferon-T signature, and/or an upregulated PD-L1 expression.

In a further embodiment the priming causes the size of the tumor to increase and/or a quantity of tumors to increase in the patient.

In a further embodiment the method comprises the immune checkpoint inhibitor comprises any one of ipilimumab; pembrolizumab; nivolumab; atezolizumab; avelumab; durvalumab; cemiplimab; tremelimumab; retifanlimab; dostarlimab; toripalimab; or combinations thereof.

In a further embodiment the method comprises the immune checkpoint inhibitor targets CTLA-4, PD-1, PD-L1, and/or LAG-3 checkpoint molecules.

In a further embodiment the method comprises administering regorafenib.

In a further embodiment the method comprises reducing the size of the tumor and/or decreasing a quantity of tumors.

In a further embodiment wherein the diameter of the tumor is decreased by 10%.

In a further embodiment the method comprises eliminating the tumor.

In yet another embodiment the method comprises a method of converting a “cold” tumor to a “hot” tumor in a patient, comprising increasing a titer of circulating CD4+ Th1 memory cells by administering an intradermal dose of a therapeutic composition to a patient, wherein the patient has a “cold” tumor and the therapeutic composition comprises activated allogeneic CD4+ T-cells; and increasing a titer of tumor-infiltrating lymphocytes at the site of the “cold” tumor by administering an intravenous dose of the therapeutic composition.

In a further embodiment the method comprises administering regorafenib to the patient.

In a further embodiment the “cold” tumor lacks tumor-infiltrating lymphocytes.

In a further embodiment the tumor comprises a metastatic colorectal cancer tumor.

In a further embodiment the tumor comprises a proficient DNA mismatch repair/microsatellite stable (pMMR/MSS) tumor.

In yet another embodiment a composition for treating metastatic colorectal cancer in a patient, comprises activated allogeneic CD4+ T-cells; and an immune checkpoint inhibitor.

In a further embodiment the immune checkpoint inhibitor comprises any one of ipilimumab; pembrolizumab; nivolumab; atezolizumab; avelumab; durvalumab; cemiplimab; tremelimumab; retifanlimab; dostarlimab; toripalimab; or combinations thereof.

In a further embodiment the immune checkpoint inhibitor targets CTLA-4, PD-1, PD-L1, and/or LAG-3 checkpoint molecules.

In a further embodiment the immune checkpoint inhibitor comprises atezolizumab

In a further embodiment the immune checkpoint inhibitor comprises avelumab.

In a further embodiment the immune checkpoint inhibitor comprises nivolumab and the composition further comprises ipilimumab.

In a further embodiment the composition comprises a multi-kinase inhibitor.

In a further embodiment the multi-kinase inhibitor comprises regorafenib.

In yet another embodiment a composition for reducing the size of an immunologically “cold” tumor in a patient, comprises an intradermal dose of activated allogeneic CD4+ T-cells; an intravenous dose of activated allogeneic CD4+ T-cells; and an intravenous dose of an immune checkpoint inhibitor.

In a further embodiment the intradermal dose comprises 0.5 mL.

In a further embodiment the intravenous dose comprises 3 mL.

In a further embodiment the immune checkpoint inhibitor comprises atezolizumab.

In a further embodiment the dose of atezolizumab comprises 1200 mg.

In a further embodiment the immune checkpoint inhibitor comprises avelumab.

In a further embodiment the dose of avelumab comprises 800 mg.

In a further embodiment the immune checkpoint inhibitor comprises nivolumab.

In a further embodiment the dose of nivolumab comprises 240 mg.

In a further embodiment the composition further comprises ipilimumab.

In a further embodiment the dose of ipilimumab comprises 1 mg/kg.

### BRIEF DESCRIPTION OF THE DRAWINGS

FIG. 1 is an image of CT scans.

FIG. 2 is an image of two graphs describing IL-12 and HSP70/HSPA1A.

FIG. 3 is an image of CT scans according to FIG. 1.

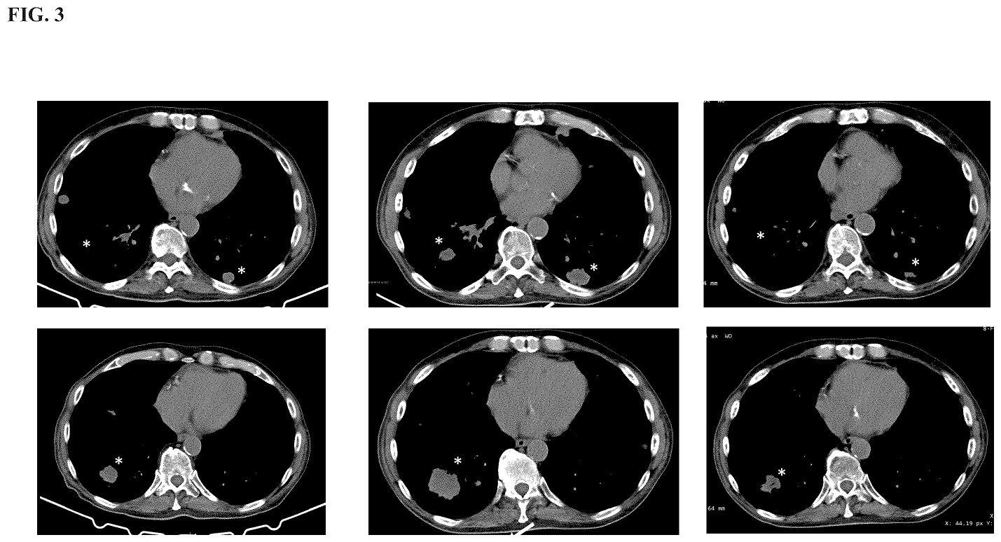

FIG. 4 is an image of CT scans according to FIG. 1.

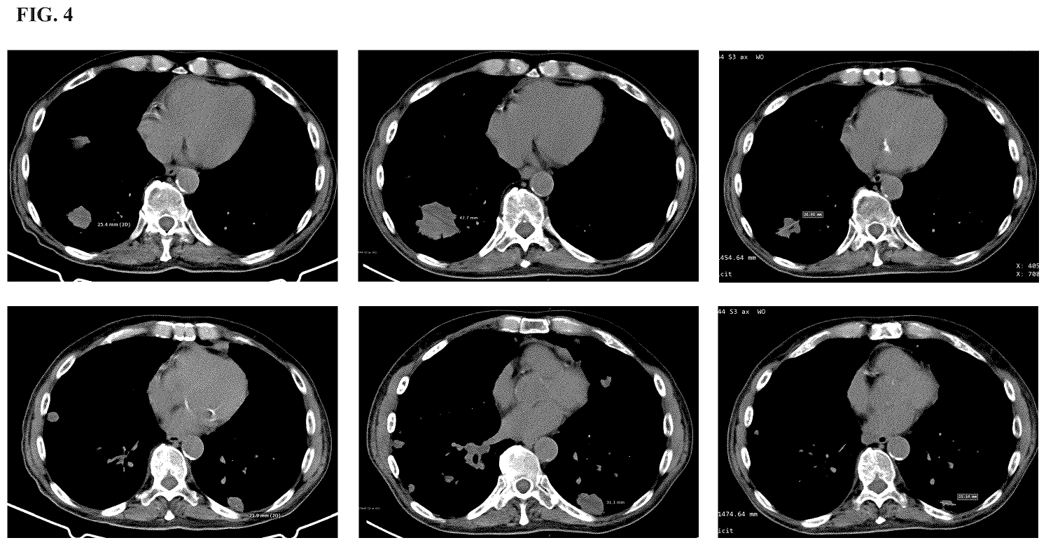

FIG. 5 is an image of CT scans and biopsies.

### DETAILED DESCRIPTION

The present disclosure relates to a therapeutic composition and methods of use thereof for treating patients with immunologically cold tumors such as metastatic colorectal cancer (mCRC) tumors. In addition, the present disclosure provides compositions and methods for converting a “cold” tumor to a “hot” tumor in a patient. In addition, the present disclosure provides compositions and methods for reducing and/or eliminating cold tumors such as metastatic colorectal cancer tumors in a patient.

The therapeutic composition is for eliciting an immunological response by the patient. The therapeutic composition comprises living cells, or components thereof. The therapeutic composition may comprise at least one highly immunogenic antigen, a molecule that delivers a signal through binding to surface CD40 receptor, and one or more inflammatory type 1 cytokines and/or chemokines. The therapeutic composition may be administered as one or more doses and/or as one or more administrations. Components of the therapeutic composition may be delivered together or individually at the same time or separately in time.

The therapeutic composition may be used and/or combined with one or more immune checkpoint inhibitors to reduce and/or eliminate a tumor. In one or more embodiments, the therapeutic composition is used with an immune checkpoint inhibitor drug targeting CTLA-4, PD-1, PD-L1, and/or LAG-3 checkpoint molecules.

In one or more embodiments, the therapeutic composition may comprise activated allogeneic CD4+ T-cells. The therapeutic composition may be combined with and/or used with one or more immune checkpoint inhibitors. For example, the therapeutic composition may be used with avelumab anti-PD-L1 monoclonal antibody. In another example, the therapeutic composition may be used with atezolizumab. In another example, the therapeutic composition may be used with regorafenib, nivolumab, and ipilimumab. The therapeutic composition and the immune checkpoint inhibitor may be administered simultaneously or sequentially to a patient.

The therapeutic composition may be used with any molecule/antibody that blocks the PD-1/PD-L1 pathway and/or any checkpoint inhibitor (e.g., anti-CTLA4, anti-LAG3) alone or in combination with another checkpoint inhibitor. The therapeutic composition may also be used with a multi-kinase inhibitor or other type of inhibitor, drug, and/or biological agent other than an immune checkpoint inhibitor.

Immune checkpoint inhibitors within the scope of this disclosure include: ipilimumab (Yervoy); pembrolizumab (Keytruda); nivolumab (Opdivo); atezolizumab (Tecentriq); avelumab (Bavencio); durvalumab (Imfinzi); cemiplimab (Libtayo); tremelimumab (Imjudo); retifanlimab (Zynyz); dostarlimab (Jemperli); and toripalimab (Loqtorzi) alone and in combination.

Other drugs within the scope of this disclosure include: regorafenib (Stivarga); TAS-102 (trifluridine and tipiracil); and fruquintinib alone and in combination. Regorafenib is an oral multi-kinase inhibitor that targets angiogenic, stromal, and oncogenic receptor tyrosine kinase (RTK). Regorafenib shows anti-angiogenic activity due to its dual targeted VEGFR2-TIE2 tyrosine kinase inhibition. TAS-102 (Lonsurf®) is an oral drug that combines trifluridine, a nucleoside metabolic inhibitor, and tipiracil, a thymidine phosphorylase inhibitor. Fruquintinib is an orally administered tyrosine kinase inhibitor (TKI) targeting vascular endothelial growth factor receptors (VEGFRs), which inhibits VEGFR phosphorylation, endothelial cell proliferation, and tubule formation.

Biological agents within the scope of this disclosure include: bevacizumab, a human monoclonal antibody (mAb) that targets vascular endothelial growth factor (VEGF); cetuximab, a chimeric human:mouse mAb against the epidermal growth factor receptor (EGFR); panitumumab, a fully human mAb that targets the extracellular domain of EGFR, alone and in combination.

In one or more embodiments, the therapeutic composition is a living, allogeneic activated Th1-like immune cell therapy derived from CD4+ T-cell precursors isolated from the blood of healthy donors. The therapeutic composition may comprise activated Th1 cells having an activated Th1 memory phenotype: CD4+, CD45RO+, CD62Llo, CD40Lhi, CD25+, IFN-γ+, and IL-4−. The activated Th1 cells can be maintained in an activated state by continuous attachment to CD3/CD28-monoclonal antibody-coated microparticles. The key effector molecules of the activated Th1 cells are the high surface expression of CD40L and the production of high amounts of inflammatory cytokines, such as interferon-gamma (IFN-γ), tumor necrosis factor-alpha (TNF-α), and granulocyte-macrophage colony stimulating factor (GM-CSF).

In one or more embodiments, the therapeutic composition comprises activated allogeneic CD4+ T-cells, which can be allogeneic activated memory CD4+ T-cells with high surface expression of CD40L and which produce IFN-γ.

The highly immunogenic antigen component of the therapeutic composition can be natural, synthetic, or recombinant proteins or peptides that have some foreign component that can make them recognizable to the human immune system. The immunogenic antigens can be, for example, allogeneic or xenogeneic protein antigens. Self-proteins that are altered to be recognized as foreign are also within the scope of this disclosure. The alteration of the self-protein can be by recombinant or chemical means or by mixing the self-protein with an adjuvant. In one embodiment, the highly immunogenic antigen is part of a living cell, preferably an allogeneic living cell, preferably a living allogeneic immune cell, more preferably an allogeneic living Th1 immune cell. Alloantigens are a preferred highly immunogenic antigen included in the therapeutic composition.

The highly immunogenic antigen(s) of the composition can be capable of being processed by professional antigen presenting cells (APC) for presentation on Major Histocompatibility Complex class I (MHC I) and/or Major Histocompatibility Complex class II (MHC II) molecules. Examples of highly immunogenic antigens may also include Keyhole limpet hemocyanin (KLH), viral proteins, bacterial protein, yeast proteins, fungal proteins, or combinations thereof.

Examples of adjuvants that can increase the immunogenicity of a protein, such as a self-protein, include agents which cause immature dendritic cells to mature to IL-12+ DC1 cells. Examples include adjuvant danger signals such as lipopolysaccharides (LPS), Bacillus Calmette-Guérin (BCG), and Toll-Like receptor agonists (e.g., TLR4 and TLR7). All highly immunogenic peptides and proteins are within the scope of this description.

The therapeutic composition can also include type I cytokines and/or chemokines. Preferred Type 1 cytokines for the therapeutic composition can include IFN-γ, IL-2, TNF-alpha, TNF-beta, GM-CSF, IL-1, IL-7, IL-15, IL-23, and IL-12 individually or in combinations thereof. Preferred chemokines for the therapeutic composition can include RANTES (Regulated upon Activation, Normal T Cell Expressed and Secreted), Macrophage Inflammatory Protein-1 alpha (MIP-1 alpha), Macrophage Inflammatory Protein-1 beta (MIP-1 beta), and Monocyte Chemoattractant Protein-1 (MCP-1) individually or in combinations thereof. The type I cytokines and/or chemokines can be exogenous and/or induced in the patient by the therapeutic composition. Exogenous cytokines and/or chemokines may be added to the therapeutic composition prior to administration to the patient.

The therapeutic composition can also include a molecule that delivers a signal through surface CD40 receptor. One preferred molecule in the therapeutic composition that delivers a signal through CD40 is immobilized CD40L. CD40L (also known as CD154) is a member of the Tumor Necrosis Factor (TNF) superfamily. CD40L can act as a co-stimulatory molecule that interacts with CD40 expressed on dendritic cells (DC) to support their maturation to an IL-12+ phenotype. CD40L is preferably immobilized by expression on a cell surface so that it provides a positive signal through CD40. Alternatively, an agonist to CD40 can be used to deliver a CD40 signal, such as a fusion protein or an anti-CD40 antibody. The components of the therapeutic composition can be delivered together or separately and in various sequences and at various points in time and are within the scope of this disclosure.

In one or more embodiments, the therapeutic composition is AlloStim®. AlloStim® is a living, allogeneic (“off-the-shelf”), non-genetically manipulated, and activated Th1-like immune cell therapy derived from CD4+ T-cell precursors isolated from the blood of healthy donors obtained from Mirror Biologics, Inc. of Wesley Chapel, Florida. AlloStim® has an activated Th1 memory phenotype: CD4+, CD45RO+, CD62Llo, CD40Lhi, CD25+, IFN-γ+, and IL-4−. AlloStim® can be maintained in an activated state by continuous attachment to CD3/CD28-monoclonal antibody-coated microparticles. The key effector molecules of AlloStim® are the high surface expression of CD40L and the production of high amounts of inflammatory cytokines, such as IFN-γ, tumor necrosis factor-alpha, and granulocyte-macrophage colony stimulating factor (GM-CSF). AlloStim® and methods of making AlloStim® are described, for example, in U.S. Pat. Nos. 7,435,592, 7,678,572, and 7,402,431, all incorporated herein by reference. Other allogeneic or xenogeneic immune cells can also be used as components in the therapeutic composition. Some of the methods of the present disclosure are described with reference to AlloStim®, but this is not meant to limit the methods to the use of AlloStim®. Other compositions may be used in the described methods.

The FDA has approved immune checkpoint inhibitor drugs targeting CTLA-4, PD-1, PD-L1, and LAG-3 checkpoint molecules for a variety of solid tumor indications, including melanoma, renal, bladder, lung, gastric, gastroesophageal junction, hepatocellular carcinoma, and head and neck cancers. However, ICIs have demonstrated only limited efficacy in metastatic colorectal cancer and other tumors considered to be immunologically cold, without resident infiltration of effector immune cells (e.g., glioblastoma, ovarian, prostate, pancreatic cancer).

An anti-CTLA4 ICI, pembrolizumab, was approved in first line mCRC and approved in combination with the anti-PD-1 ICI, nivolumab, for a subset of mCRC patients that have deficient DNA mismatch repair/microsatellite instability-high (dMMR/MSI-H) status. However, this dMMR/MSI-H subset constitutes only ˜5% of mCRC patients, while the remaining ˜95% that present with pMMR/MSS status do not respond to ICI. Most dMMR/MSI-H status tumors are considered immunologically “hot” tumors, while pMMR/MSS status tumors are considered to be “cold”. ICI have demonstrated greater efficacy in hot tumors, characterized by an inflamed phenotype, including a high level of infiltrating T-cells and NK cells, an IFN-7 signature, and upregulated PD-L1 expression, while cold tumors have an absence of tumor-infiltrating lymphocytes.

While dMMR/MSI-H mCRC patients are more responsive to ICI therapy, approximately 50% are refractory. Resistance to ICI responsiveness, regardless of MMR/MSI status, is correlated with tumor mutational burden. Since somatic mutations can encode immunogenic neoantigens, high tumor mutational burden is believed to be more likely to prime for infiltrating tumor-specific effector immune cells. Consistent with this, the dMMR/MSI-H patients that present with lower tumor mutational burden values have been shown to be the non-responders, whereas patients with the highest tumor mutational burden values tend to obtain benefit from ICI, particularly with anti-CTLA-4/PD-1 combination ICI immunotherapy. The general cut-off between “high” and “low” tumor mutational burden is about 10 mutations per Mb.

The therapeutic composition described herein is an experimental allogeneic immunomodulatory cell therapy designed to convert “cold” metastatic tumor lesions to “hot” inflamed lesions. After immunotherapy with the therapeutic composition, this cold to hot inflammatory mechanism can make it difficult to distinguish between pseudoprogression and actual progression on restaging CT scans, as inflamed metastatic lesions can appear larger and occult disease can appear as small new lesions.

To explore whether radiological progression after immunotherapy with the therapeutic composition is due to immune-flare or disease progression, a short course of a combination ICI therapy was administered to a pMMR/MSS chemotherapy-refractory metastatic colorectal cancer patient enrolled in a STIMVAX Phase IIb clinical study that was presented with radiological progression after AlloStim® immunotherapy. The rationale was that an accelerated response to ICI should occur if the lesions were inflamed, while if the enlarged lesions were due to disease progression there would not be a response. Here a rapid, significant reduction in tumor burden is reported in response to ICI administration in an AlloStim® primed pMMR/MSS mCRC patient with retroperitoneal and lung metastases. Immune checkpoint inhibitor (ICI)-based regimens have not previously shown meaningful positive outcomes in proficient DNA mismatch repair/microsatellite stable (pMMR/MSS) metastatic colorectal cancers. As an example, a rare objective response is reported in a pMMR/MSS heavily pre-treated metastatic colorectal cancer patient after a short course of an immune checkpoint inhibitor combination after first being primed with an experimental immunomodulatory cell therapy drug, AlloStim®, designed to convert immunologically “cold” tumors to “hot” tumors, and a short, low dose course of regorafenib.

The STIMVAX protocol provides for three monthly cycles of weekly administration of a therapeutic composition designed to increase circulating memory Th1/Th2 ratio, activate circulating memory T cells and NK cells, which in turn causes their extravasation to tumor sites. The anti-tumor effects are correlated with the establishment of an IFN-γ dominated microenvironment. The systemic tumor infiltration mechanism serves to convert immunologically cold tumors to hot tumors. The modulation of the tumor microenvironment can also counter-regulate tumor-mediated immune suppression.

The STIMVAX dosing pattern is designed to create self-amplifying waves of memory allo-specific Th1/CTL cells. For example, the completely mismatched AlloStim® cells are rejected by the host upon administration and do not persist past 24 h. Intradermal injections cause allo-specific Th1 immunity. Intravenous infusions are designed to cause the rejection in the blood and subsequent release of non-toxic inflammatory cytokines. The cytokine release acts to non-specifically activate circulating memory allo-specific Th1 T-cells and also activate NK cells. These activated effector cells extravasate and traffic to tumor lesions, converting the lesions from immunologically “cold” tumors to “hot” tumors. The intratumoral activated NK cells mediate immunological tumor cell death in the context of inflammatory cytokines produced by the infiltrating activated allo-specific Th1 cells. This creates the necessary conditions for in-situ vaccination and dys-regulation of immune suppressor circuits in the tumor microenvironment. The in-situ tumor lysis occurs in the context of released endogenous danger signals and intracellular tumor-specific antigen chaperone proteins, especially heat shock proteins (HSP). The endogenous HSP released after tumor lysis are known to chaperone tumor neoantigens which are then processed by resident dendritic cells (DC) in the context of released “danger signals” and the inflammatory cytokine environment. Following local processing of released HSP by DC in the tumor microenvironment, the DC mature and traffic to the draining lymph nodes priming a tumor-specific immune response. Thereafter, after each subsequent intradermal-intravenous cycle, the proportion of circulating memory T-cells that are tumor specific and allo-specific increases. Therefore, after each intravenous infusion the quantity of memory cells that extravasate and infiltrate tumor lesions increases, which can convert immunologically-cold tumors to hot tumors. This mechanism using an “off-the-shelf” cell therapy thus can create a tumor-specific immune response customized to each patient's own tumor.

Each dose of a therapeutic composition may be between about 1×10⁶ and about 1×10¹⁰ cells. In one embodiment, an intradermal dose of a therapeutic composition is at least about 1×10⁶ cells. For example, a dose of a therapeutic composition may be 0.5 mL of 1×10⁷ cells composition. Dosages outside this range that can primarily generate the desired immune response are also within the scope of this description.

Intradermal injections of the therapeutic composition can “prime” a patient to become immune to the alloantigen in the therapeutic composition. Multiple intradermal injections can increase the number of Th1 memory cells specific for the alloantigens in the circulation of the host, which in turn changes the Th1/Th2 balance. In one embodiment, an intradermal injection can include about 1×10⁶ to about 1×10⁷ cells of AlloStim®. Furthermore, a dose may be about 0.5 mL.

The intradermal dosing is preferably repeated multiple times to build up the number of circulating allo-specific Th1 memory cells. After the first dose, each subsequent dose of the therapeutic composition may be administered at least 3 days after administration of the previous dose. In one embodiment, the therapeutic composition may be administered intradermally and can be repeated about 3-6 times and about 3-10 days apart.

In one or more embodiments, an intravenous dose is administered to cause the activation of host immune cells (both innate and adaptive) and their extravasation to sites of inflammation, including tumor locations. In one or more embodiments, an intravenous dose of the therapeutic composition may include about 1×10⁷ to 1×10⁹ cells, or about 5×10⁷ to 1×10⁸ cells. The intravenous infusions may be repeated several times, for example, on a monthly basis.

In one or more embodiments, AlloStim®-mediated intratumoral type I cytokine production by infiltrating activated T-cells and NK cells, including IL-12 and IFN-γ, is believed to: upregulate MHC-I on tumor cells making them susceptible to CD8+ T-cell recognition; cause maturation of dendritic cells to DC1 (IL-12+ CD80/86 positive, MHC I and MHC II positive); convert M2 macrophages to M1; and, release neoantigens into the tumor microenvironment. The release of neoantigens into an inflammatory tumor microenvironment creates the conditions for in-situ vaccination where immature dendritic cells mature to type I dendritic cells (DC1), process the released chaperoned neoantigens, and migrate to the draining lymph nodes, resulting in a patient-specific anti-tumor adaptive immune response.

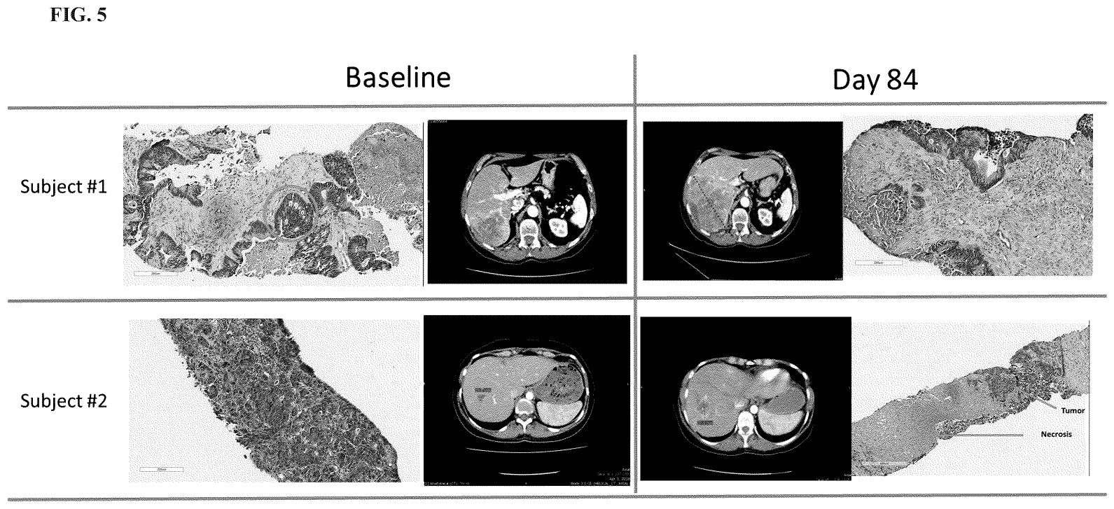

In previous clinical studies, the inflammatory mechanism of AlloStim® almost always caused post-treatment CT scan images to be read as progressive disease, with systemic increases in size of existing tumor lesions and often the appearance of new, small lesions (especially in lungs). However, this progressive disease determination did not always correlate with the clinical status of the patient or with overall survival (see FIG. 5). As the systemic increase in target lesion size could be due to peritumoral inflammation that occurs when tumor lesions convert from cold to hot and new small lesions could be the result of inflammation of occult disease, it is difficult to distinguish tumor progression from pseudoprogression using CT scan imaging after AlloStim® experimental treatment.

Pseudoprogression after immunotherapy has been observed in patients with various tumor types and is thought to be due to transient immune cell infiltration into the tumor. The phenomenon of pseudoprogression has led to modification of the RECIST 1.1 evaluation criteria. The understanding that tumor growth by RECIST does not necessarily translate to disease progression in patients treated with immunotherapy has also led to the development of immune-related response criteria (irRC) to better surveil these patients.

However, it is still considered challenging to distinguish radiological progression from pseudoprogression and, consequently, to define the best management for these patients. In addition, a new category of “hyper-progression” and dissociated atypical responses have also been described after immunotherapy. These issues have resulted in some subjects being prematurely removed from immunotherapy clinical trials.

In the CheckMate 142 clinical trial, nivolumab (3 mg/kg) plus low-dose ipilimumab (1 mg/kg) provided durable clinical benefit, and a manageable safety profile in patients with previously treated dMMR/MSI-H metastatic CRC. Due to the improved safety profile of the lower dose ipilimumab in this combination, this regimen was tested in a chemotherapy-refractory mCRC pMMR/MSS patient presenting with radiological progression after 3 cycles of AlloStim® experimental immunotherapy.

If the radiological progression was due to inflammation within the tumor lesions (hot tumors), a short course of ICI immunotherapy should result in a rapid reduction in tumor burden, due to release of suppression of resident infiltrating effector immune cells. On the other hand, if the enlarged and increased number of lesions were due to true progression and no infiltrating effector immune cells were present, either no response or further progression would be expected to be observed.

### Prophetic Example—Atezolizumab

The protocol will provide fourth-line experimental treatment with AlloStim® administered weekly in 3-21 day cycles each cycle consisting of 3 weekly intradermal (ID) doses followed the last week with an intravenous (IV) dose (3 cycles=Days 0-77). A restaging CT scan will be conducted on day 84 and will be compared to the baseline CT scan by RECIST 1.1. Most scans are expected to be read as radiological progression upon restaging after AlloStim® priming but is believed to be “pseudoprogression” due to the tumor inflammatory mechanism of action of the STIMVAX protocol whereby “cold” tumors convert to “hot” tumors. After AlloStim® priming, on days 91-161, a combination of AlloStim® IV boosters with anti-PD-L1 checkpoint therapy (atezolizumab 1200 mg q/3 weeks) are scheduled. Restaging CT scan at day 175 will be compared to day 84 and baseline. Thereafter, subjects will be followed for survival. Experimental end-points include longitudinal ctDNA levels and experimental plasma biomarkers (research blood).

AlloStim® is the investigational product (IP) for this study. The active ingredient in AlloStim® is viable, activated polyclonal CD4+ memory Th1-like T-cells that express high density CD40L and Th1 helper (IFN-gamma+, IL-4−) properties. In final formulation, AlloStim® is a combination biological drug (somatic cell therapy) and medical device (microbeads). Source cells are CD4+ T-cells positively selected from buffy coat material prepared pursuant to 21 CFR 1271 from known healthy, screened, paid blood donors. These CD4+ T-cells are differentiated and expanded ex-vivo in a 9-day culture process to become Th1-like effector cells (“T-Stim™”) T-Stim™ batches are stored in liquid nitrogen as an intermediate product. When required in the clinic, T-Stim™ cells are thawed, activated with microbeads for 4 hours causing differentiation of the cells to an activated Th1 memory cell phenotype, which are then harvested, washed and formulated in media for infusion and aliquoted into vials and then frozen in liquid nitrogen. This formulated combination of cells with microbeads attached is called AlloStim®. The attached microbeads have bound mouse anti-human CD3 and CD28 monoclonal antibodies (mAbs) (either Dynabeads® ClinExVivo™ CD3/CD28 or Miltenyi T cell activation beads) which activate the somatic cells and serve to maintain this activated state upon infusion or injection.

Research blood will be collected from subjects for analysis of sera for cytokines (such as IL-12-p70, IFN-γ, and TNF-α) and experimental biomarkers, such as sHSP70, sCD163. AlloStim® is designed to activate a Th1 immune response. Measuring IL-12, TNF-α, and IFN-7 in a subject's sera can provide evidence to support activation and sustainability of a Th1 immune response activated by AlloStim®. In addition, chronic inflammatory adverse events could be attributed to AlloStim® by following these cytokines, especially TNF-α.

AlloStim® is formulated T-Stim™ cells that have been activated by adding CD3/CD28-conjugated microbeads and packaged in a cryovial. AlloStim® is prepared by thawing frozen, QC released T-Stim™ cells and incubating the cells for 4 hours with anti-CD3/CD28 microbeads. The beads are mixed with the cells at a 1:1 bead to cell ratio for Dynal beads and 1:4 bead to cell ratio for Miltenyi beads. After 4 hours, the cells with the beads attached are harvested, washed and suspended in formulation buffer containing PlasmaLyteA® supplemented with 1% human serum albumin with 2% DMSO. The formulated cells with the beads still attached are then loaded in single dose cryovials and stored at −180° C. in LN2. The vials are shipped to central storage and/or clinical sites in a LN2 dry shipper. The IP drug AlloStim® is formulated in cryovials as follows:
Intradermal (ID) dose: 0.5×10⁷ cells with beads in 0.5 mL in a 3 mL vial
Intravenous (IV) dose: 3.0×10⁷ cells with beads in 3 mL in a 5 mL vial

Intradermal Injection of AlloStim®: Each ID injection contains 0.5×10⁷ AlloStim® cells formulated in 0.5 mL of 1% human serum albumin in PlasmaLyteA and 2% DMSO. One pre-formulated 3 mL vial of AlloStim® will be delivered to the study site per subject for the ID injection days. ID Injections shall be administered into the forearm (abdomen and deltoid areas can also be used) For the ID injection, the needle should be at a shallow angle, approximately 15 degrees or less (in the dermal layer of the skin).

Intravenous Infusion of AlloStim®: Each IV dose contains 3.0×10⁷ AlloStim® cells formulated in 3 mL of 1% human serum albumin in PlasmaLyteA with 2% DMSO. AlloStim® is administered as an IV push. Subjects should be hydrated with 500 mL NS or D5W before and after infusion.

### Priming:

Cycle 1: Day 0—0.5 mL ID AlloStim®+ctDNA+research blood;
   Day 7—0.5 mL ID AlloStim®;
   Day 14—0.5 mL ID AlloStim®;
   Day 21—3 mL IV AlloStim®.
Cycle 2: Day 28—0.5 mL ID AlloStim®+research blood;
   Day 35—0.5 mL ID AlloStim®;
   Day 42—0.5 mL ID AlloStim®;
   Day 49—3 mL IV AlloStim®.
Cycle 3: Day 56—0.5 mL ID AlloStim®+research blood;
   Day 63—0.5 mL ID AlloStim®;
   Day 70—0.5 mL ID AlloStim®;
   Day 77—3 mL IV AlloStim®.
Day 84—CT Scan

### AlloStim®+Atezolizumab:

Day 91—3 mL IV AlloStim®+research blood+ctDNA;
Day 98—1200 mg IV Atezolizumab;
Day 105—no treatment;
Day 112—3 mL IV AlloStim®;
Day 119—1200 mg IV Atezolizumab;
Day 126—no treatment;
Day 133—3 mL IV AlloStim®;
Day 140—1200 mg IV Atezolizumab;
Day 147—no treatment;
Day 154—3 mL IV AlloStim®;
Day 161—1200 mg IV Atezolizumab;
Day 168—research blood+ctDNA.
Day 175—CT Scan

### Prophetic Example—Avelumab

The protocol will provide fourth-line experimental treatment with AlloStim® administered weekly in two 21-day cycles (Priming Phase) each cycle consisting of 3 weekly ID doses followed the last week with an IV dose (2 cycles=Days 0-49). A restaging CT scan will be conducted on Day 56 and will be compared to the Baseline CT scan by RECIST 1.1. Most scans are expected to be read as radiological progression upon restaging after AlloStim® priming, but in most subjects this finding is believed to be “pseudoprogression” due to the tumor inflammatory mechanism of action of AlloStim® (immunologically “cold” tumors convert to “hot” inflamed tumors). After AlloStim® priming, on Days 63-105, a Combination Phase will be initiated with AlloStim® IV boosters and avelumab (800 mg Q2W). Restaging CT scan at Day 112 will be compared to Day 56 (end of Priming Phase) and Baseline. For subjects that remain clinically stable, they can advance to an Expansion Phase of continued alternating 2 week dosing of AlloStim® and avelumab (Day 119-Day 161). A restaging CT scan will be conducted at Day 168 for all subjects (regardless of whether they participated in the Expansion Phase) to be compared to Baseline, Day 56, and Day 112 scans. Thereafter, subjects are followed for survival. Experimental end-points include experimental plasma biomarkers (research blood).

Avelumab, also known by the brand name Bavencio, is a human IgG1 monoclonal antibody that specifically binds to the checkpoint inhibitor molecule PD-L1, preventing the interaction between PD-L1 and the inhibitory T-cell receptor, PD-1. Avelumab is cleared by US FDA for: (1) the treatment of adults and pediatric patients 12 years and older with metastatic Merkel cell carcinoma (MCC); (2) the maintenance treatment of patients with locally advanced or metastatic urothelial carcinoma (UC) that has not progressed with first-line platinum-containing chemotherapy; and (3) the treatment of patients with locally advanced or metastatic UC who have disease progression during or following platinum-containing chemotherapy or have disease progression within 12 months of neoadjuvant or adjuvant treatment with platinum-containing chemotherapy. Avelumab will be used off-label in this protocol.

AlloStim® will be the investigational product (IP) for this study. The active ingredient in AlloStim® is viable, activated polyclonal CD4+ memory Th1-like T-cells that express high density CD40L and Th1 helper (IFN-gamma+, IL-4−) properties. In final formulation, AlloStim® is a combination biological drug (somatic cell therapy) and medical device (microbeads). Source cells are CD4+ T-cells positively selected from buffy coat material prepared pursuant to 21 CFR 1271 from known healthy, screened, paid blood donors. These CD4+ T-cells are differentiated and expanded ex-vivo in a 9-day culture process to become Th1-like effector cells (“T-Stim™”) T-Stim™ batches are stored in liquid nitrogen as an intermediate product. When required in the clinic, T-Stim™ cells are thawed, activated with microbeads for 4 hours causing differentiation of the cells to an activated Th1 memory cell phenotype, which are then harvested, washed and formulated in media for infusion and aliquoted into vials and then frozen in liquid nitrogen. This formulated combination of cells with microbeads attached is called AlloStim®. The attached microbeads have bound mouse anti-human CD3 and CD28 monoclonal antibodies (mAbs) (either Dynabeads® ClinExVivo™ CD3/CD28 or Miltenyi T cell activation beads) which activate the somatic cells and serve to maintain this activated state upon infusion or injection.

The mechanism of action of AlloStim® can convert metastatic lesions from “cold” to “hot.” The increase in immune cell infiltration can make the tumors appear larger on a CT scan. In addition, previously occult lesions may become inflamed and appear as new lesions. For these reasons, radiological progression by RECIST criteria is difficult to distinguish pseudoprogression from actual progression. The cold colorectal cancer metastatic MSS/pMMR lesions do not respond to checkpoint inhibitor immunotherapy. The hypothesis is that if the lesions are inflamed with activated Th1 cells, there will be an increase in intra-tumoral interferon-gamma production. However, interferon-gamma is known to increase the expression of PD-L1 in tumor lesions. High expressing PD-L1 tumors are known to be responsive to anti-PD-L1 immunotherapy. Therefore, the hypothesis is that initial AlloStim® priming followed by anti-PD-L1 immunotherapy could result in objective tumor responses that could then prevent premature discontinuation from the clinical trial and provide for possible benefit to subjects.

Study Population: Adult patients ≥18 years and ≤80 years with adequate performance status (ECOG 0-1) and adequate organ function with microsatellite stable (MSS), proficient mismatch repair (pMMR) metastatic, histologically confirmed, adenocarcinoma of the rectum or colon that has been previously treated with at least two lines of active chemotherapy.

AlloStim® is formulated T-Stim™ cells that have been activated by adding CD3/CD28-conjugated microbeads and packaged in a cryovial. AlloStim® is prepared by thawing frozen, QC released T-Stim™ cells and incubating the cells for 4 hours with anti-CD3/CD28 microbeads. The beads are mixed with the cells at a 1:1 bead to cell ratio for Dynal beads and 1:4 bead to cell ratio for Miltenyi beads. After 4 hours, the cells with the beads attached are harvested, washed and suspended in formulation buffer containing PlasmaLyteA® supplemented with 1% human serum albumin with 2% DMSO. The formulated cells with the beads still attached are then loaded in single dose cryovials and stored at −180° C. in LN2. The vials are shipped to central storage and/or clinical sites in a LN2 dry shipper. The IP drug AlloStim® is formulated in cryovials as follows:
Intradermal (ID) dose: 0.5×10⁷ cells with beads in 0.5 mL in a 3 mL vial
Intravenous (IV) dose: 3.0×10⁷ cells with beads in 3 mL in a 5 mL vial

Intradermal (ID) Injections of AlloStim®: Each ID injection contains 0.5×10⁷ AlloStim® cells formulated in 0.5 mL of 1% human serum albumin in PlasmaLyteA and 2% DMSO. One pre-formulated 3 mL vial of AlloStim® will be delivered to the study site per subject for the ID injection days. ID Injections shall be administered into the forearm (abdomen and deltoid areas can also be used) For the ID injection, the needle should be at a shallow angle, approximately 5 degrees or less (in the dermal layer of the skin).

Intravenous (IV) Infusions of AlloStim®: Each IV dose contains 3.0×10⁷ AlloStim® cells formulated in 3 mL of 1% human serum albumin in PlasmaLyteA with 2% DMSO. AlloStim® will be administered as an IV push. Subjects should be hydrated with 500 mL NS or D5W before and after infusion. Pre-medication with 25-50 mg diphenhydramine PO/IV, an H-2 blocker, or 25-50 mg PO indomethacin (or equivalent NSAID) during or after the infusion is allowed if medically indicated.

Intravenous (IV) Infusions of Avelumab: Avelumab is provided in 10 mL single dose vials containing 200 mg avelumab (20 mg/mL) for infusion in a single-dose vial. Each infusion requires 800 mg dose (4 vials). Avelumab will be administered as an intravenous infusion over 60 minutes every 2 weeks until disease progression or unacceptable toxicity. Before the first four infusions of avelumab, patients are recommended to be premedicated with an antihistamine and acetaminophen to reduce the risk of infusion-related reactions.

### Priming: AlloStim® Only

Cycle 1: Day 0—0.5 mL ID AlloStim®+research blood+autoimmune panel;
   Day 7—0.5 mL ID AlloStim®;
   Day 14—0.5 mL ID AlloStim®;
   Day 21—3 mL IV AlloStim®.
Cycle 2: Day 28—0.5 mL ID AlloStim®+research blood;
   Day 35—0.5 mL ID AlloStim®;
   Day 42—0.5 mL ID AlloStim®;
   Day 49—3 mL IV AlloStim®.
   Day 56—CT Scan

### Combination: AlloStim®+Avelumab

Day 63—3 mL IV AlloStim®+research blood;
Day 70—800 mg IV avelumab;
Day 77—3 mL IV AlloStim®;
Day 84—800 mg IV avelumab;
Day 91—3 mL IV AlloStim®;
Day 98—800 mg IV avelumab;
Day 105—Clinic Visit/Research Blood.
Day 112—CT Scan

### Expansion: AlloStim®+Avelumab (for Clinically Stable Subjects)

Day 119—3 mL IV AlloStim®;
Day 126—800 mg IV avelumab;
Day 133—3 mL IV AlloStim®;
Day 140—800 mg IV avelumab;
Day 147—3 mL IV AlloStim®;
Day 154—800 mg IV avelumab;
Day 161—Clinic Visit/Research Blood.
Day 168—CT scan

### Example—Regorafenib+Nivolumab+Ipilimumab

A 69-year-old patient presented with blood in the stool. Upon workup, was found to have a rectal mass and subsequently underwent low anterior resection. 2 of 24 nodes were found to be positive for adenocarcinoma disease with initial staging of pT2N1b. The patient was treated with adjuvant FOLFOX and achieved a complete response. Disease recurred, and the patient underwent transanal resection plus radiation therapy. A right inguinal node was identified as metastatic adenocarcinoma and the patient received additional radiation therapy. The patient subsequently presented with enlarged retroperitoneal nodes and was treated with 22 cycles of FOLFIRI plus Avastin. The patient was then treated again with FOLFOX but developed a reaction to oxaliplatin.

A CT Scan showed appearance of innumerable bilateral pleural parenchymal lung nodules significantly increased in size and number from prior examination, and significant worsening of mediastinal and hilar lymphadenopathy consistent with progressive metastatic disease. No suspicious liver lesions were found, and stable non-specific retroperitoneal adenopathy was also noted. Target lesions were identified in the lungs. The retroperitoneal disease was too small (<15 mm) to be included in the RECIST 1.1 evaluation.

Patient consented to receive AlloStim® experimental immunotherapy as part of the STIMVAX Phase IIB clinical trial (NCT04444622). Eligible patients had histologically confirmed pMMR/MSS adenocarcinoma of the colon or rectum; received all available standard systemic therapies (fluoropyrimidines, oxaliplatin, irinotecan, and bevacizumab; cetuximab or panitumumab if RAS wild-type tumors); were aged 18 years or older; had adequate organ function; Eastern Cooperative Oncology Group (ECOG) performance status of 0 or 1, and measurable disease. The study was conducted in accordance with the Declaration of Helsinki and Good Clinical Practice Guidelines after approval by a central institutional review board (IRB) and the ethics board at each institution where applicable.

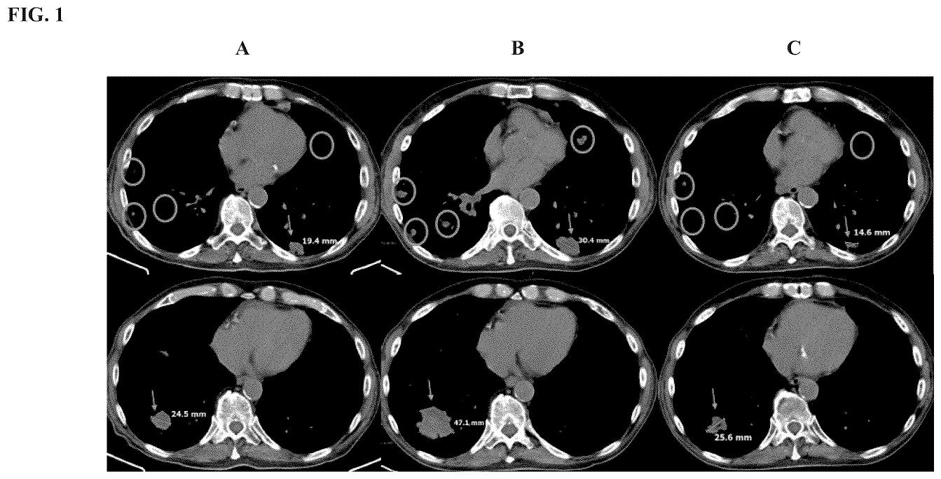

The dosing and procedure schedule is shown in Table 1 and longitudinal changes in lung target lesions from CT scans are shown in FIG. 1. Three 28-day cycles of weekly AlloStim® immunotherapy were administered per protocol. The CT scan comparison from baseline (Scan A) to completion of the AlloStim® three cycles (Scan B) demonstrated progressive disease by RECIST 1.1 evaluating target lesions in the lungs and retroperitoneum (sum of diameters of target lesions=+73%) with increased non-target retroperitoneal adenopathy and innumerable enlarging and new thoracic nodules.

Three cycles of weekly AlloStim® immunotherapy were administered per protocol. AlloStim® was administered at a dose of 10⁷ cells for the intradermal and intravenous doses. Each intradermal dose was administered at 0.5 mL, and each intravenous dose was administered at 3 mL.
Cycle 1: Day 0—0.5 mL ID;
   Day 7—0.5 mL ID;
   Day 14—0.5 mL ID;
   Day 21—0.5 mL ID;
   Day 28—0.5 mL ID+3 mL IV.
Cycle 2: Day 42—0.5 mL ID;
   Day 49—0.5 mL ID;
   Day 56—0.5 mL ID;
   Day 63—0.5 mL ID;
   Day 70—0.5 mL ID+3 mL IV.
Cycle 3: Day 84—0.5 mL ID;
   Day 91—0.5 mL ID;
   Day 98—0.5 mL ID;
   Day 105—0.5 mL ID;
   Day 112—0.5 mL ID+3 mL IV.

A short and reduced dose of regorafenib was administered at 40 mg bid (80 mg/day total). Regorafenib dose was increased with alternating 40 mg bid (80 mg/day total) and 40 mg tid (120 mg/day total). A short course of nivolumab (240 mg) and low dose ipilimumab (1 mg/kg) was administered. Then, no treatment was administered, during which time a tapering dose of oral prednisone beginning at 60 mg/day was administered for treatment of colitis (adverse effect from the combination ICI immunotherapy), a restaging CT scan was then obtained.

TABLE 1
SCHEDULE OF PROCEDURES

| PROCEDURE | ROUTE (10⁷ cells) | |
|---|---|---|
| CT Scan A | | |
| AlloStim | ID (0.5 mL) | |
| AlloStim | ID (0.5 mL) | |
| AlloStim | ID (0.5 mL) | |
| AlloStim | ID (0.5 mL) | |
| AlloStim | ID (0.5 mL) + IV (3 mL) | |
| AlloStim | ID (0.5 mL) | |
| AlloStim | ID (0.5 mL) | |
| AlloStim | ID (0.5 mL) | |
| AlloStim | ID (0.5 mL) | |
| AlloStim | ID (0.5 mL) + IV (3 mL) | |
| AlloStim | ID (0.5 mL) | |
| AlloStim | ID (0.5 mL) | |
| AlloStim | ID (0.5 mL) | |
| AlloStim | ID (0.5 mL) | |
| AlloStim | ID (0.5 mL) + IV (3 mL) | |
| CT Scan B | | |
| regorafenib | 40 mg bid (80 mg/day) | |
| regorafenib | 80 mg day 1/120 mg day 2 | |
| | (alternating) | |
| Nivolumab | 240 | mg |
| Ipilimumab | 1 | mg/kg |
| Nivolumab | 240 | mg |
| Nivolumab | 240 | mg |
| Ipilimumab | 1 | mg/kg |
| Prednisone | 60 mg/day taper | |
| CT Scan C | | |

Scan C demonstrated a partial response by RECIST 1.1 criteria in comparison to the post-AlloStim® Scan B (change in sum of diameters of target lesions=−48%) with the previously observed innumerable non-target thoracic lesions and retroperitoneal adenopathy uniformly decreased in size and number. Comparison to Scan A (baseline) was read as stable disease with a 10% decrease in sum of diameters of target lesions.

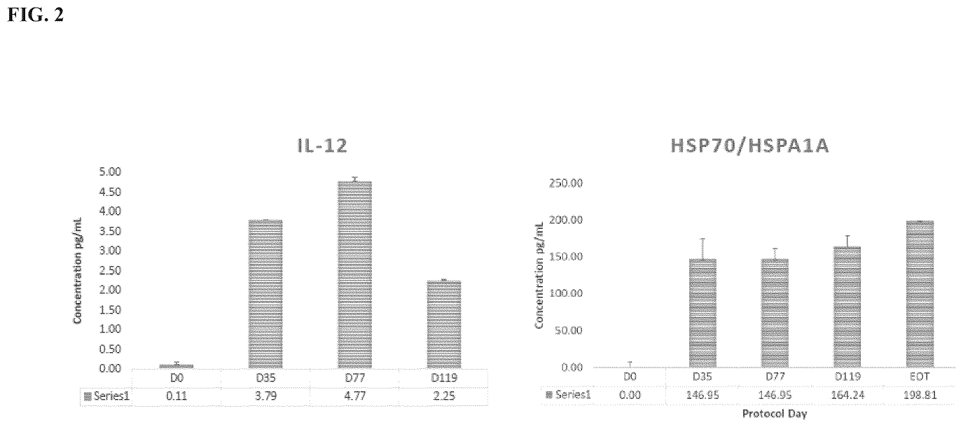

Serum levels of IL-12 levels were negligible prior to AlloStim® administration. After AlloStim® dosing, IL-12 became detectable after the first cycle and remained detectible over the 3 cycles of AlloStim® administration. Soluble heat shock protein (HSP)-70 was also negligible at baseline but was elevated throughout the experimental immunotherapy dosing (see FIG. 2).

### Discussion

The experiment demonstrates a rapid tumor debulking response after a short course of combination ICI immunotherapy in a heavily pre-treated pMMR/MSS mCRC patient presenting with 60 radiological progressions after third-line experimental AlloStim® immunotherapy and a short course of low dose regorafenib.

It is further considered whether the ICI immunotherapy alone or in combination with previous AlloStim®, or in combination with previous regorafenib alone, or with prior AlloStim® and regorafenib together was most likely responsible for eliciting this rare objective response in a cold tumor indication.

It is unlikely that the combination ICI immunotherapy is solely responsible for the observed response. ICI-based regimens, both as monotherapy and as combination therapies, have not previously shown any meaningful positive outcomes in pMMR/MSS colorectal cancers.

For example, an initial phase II study assessed the efficacy of tremelimumab, a monoclonal antibody against CTLA4, in patients with treatment-refractory mCRC, which resulted in no improvement post-treatment (J Clin Oncol 2010, 28:3485-3490). Furthermore, two phase I studies of anti-PD-1 (N Engl J Med 2012, 366:2443-2454) and anti-PD-L1 (N Engl J Med 2012, 366:2455-2465) in previously treated mCRC patients produced no responses. ICI regimens also failed as maintenance therapy after first line therapy in the MODUL study (Mol Cancer Res 2018, 16:805-812).

In general, ICI immunotherapy combining CTLA-4 and PD-L1 inhibitors have also shown very limited clinical benefit in patients with non-selected mCRC. A rare partial response (1/119) was reported in a randomized phase 2 clinical trial which evaluated the efficacy of combination durvalumab (anti-PD-L1) and tremelimumab (anti-CTLA-4) in patients with advanced refractory mCRC. In this study, 119 patients were assigned to the treatment group and 61 patients were assigned to best supportive care alone. Patients in the treatment group received a median of 12 weeks of durvalumab and 12 weeks of tremelimumab (JAMA Oncol 2020, 6:831-838), while in the present example only 5 weeks of ICI combination therapy was administered.

The phase II KEYNOTE-016 trial was performed to evaluate the clinical efficacy of single agent pembrolizumab in patients with pMMR/MSS mCRC, dMMR/MSI-H mCRC and or dMMR/MSI-H non-CRC. No responses were noted in 18 patients in the pMMR/MSS mCRC group (N Engl J Med 2015, 372:2509-2520). In a clinical study which included 59 pMMR/MSS mCRC patients treated with ICI beyond radiological progression by RECIST 1.1, no patient demonstrated subsequent radiographical tumor shrinkage at a median of 42 days (J Immunother 2018, 41:284-291).

It has been reported that a small subset (˜2%) of patients with pMMR/MSS colorectal cancer with a mutation in POLE and POLD1 enzymes and those without liver metastases have a higher chance of a response to ICI immunotherapy (J Gastrointest Cancer 2023, 54:1017-1030). While the patient in the present example did not have POLE or POLD1 mutations, no liver metastases were present. Therefore, it is possible this patient was more susceptible to ICI immunotherapy but seems unlikely that the short course of combination ICI alone was solely responsible for the extensive tumor debulking observed.

ICI strategies in combination with other drugs or procedures are under investigation, including evaluations of ICI in combinations with chemotherapy, radiotherapy, vascular endothelial growth factor (VEGF)/VEGF receptor (VEGFR) inhibitors, mitogen-activated protein kinase (MEK) inhibitors, and signal transducer and activation of transcription 3 (STAT3) inhibitors (Cancer Immunol Immunother 2023, 72:3875-3893). However, these combination approaches have yet to demonstrate any significant anti-tumor activity in the clinical setting (Cancers (Basel) 2023, 15; Gastroenterol Rep (Oxf) 2020, 8:11-24; World J Gastroenterol 2019, 25:3920-3928).

Regorafenib is approved for third line mCRC based on the results of the CORRECT trial which demonstrated only a 1.4 month increase in the median survival compared to a placebo control (6.4 months vs. 5 months). In the Phase II TEXCAN trial, no objective responses were reported in 35 mCRC patients after 2 months of treatment with regorafenib according to RECIST 1.1, Choi, and modified Choi (Cancer Imaging 2019, 19:85). Therefore, it seems unlikely that the prior short course of low dose regorafenib alone could be responsible for the rare objective response reported here.

Regorafenib is a multi-kinase inhibitor that targets several receptor tyrosine kinases involved in angiogenesis and metastases (VEGFR1, VEGFR2, VEGFR3, FGFR1, FGFR2, TIE2, PDGFRs), oncogenesis (KIT, RET, RAF1), and tumor immunity (CSF1R). While regorafenib does not directly convert cold tumors to hot tumors, regorafenib is believed to possibly contribute to shifting the tumor microenvironment toward a more immune-responsive state. This constellation of mechanisms suggests that regorafenib could potentially be a combination partner for ICIs (Cancer Treat Rev 2022, 110:102460).

There are mixed results on the combination of regorafenib with ICI in clinical trials. Regorafenib in combination with PD-1 antibody as a third line mCRC therapy has been evaluated in several studies. For example, 24 patients with MSS mCRC were included in the REGONIVO study. In this study, regorafenib was administered at 80-160 mg once daily for 21 days on and 7 days off together with nivolumab at 3 mg/kg every 2 weeks. A 33.3% objective response rate was reported with this regimen (J Clin Oncol 2020, 38:2053-2061). However, this promising activity has not been observed in other studies.

In a single site study, 18 mCRC patients treated with a combination of regorafenib and nivolumab, no objective responses were observed. In this study, 13 patients (69%) had progressive disease, and the median progression-free survival was only 2 months. Four out of five patients in this study evaluated with stable disease occurred in patients without liver metastases, whereas a short disease stabilization was seen in 1 of 14 patients with history of liver metastases (Oncologist 2020, 25:e1188-e1194).

In another study in MSS mCRC patients, a combination of regorafenib and toripalimab, an anti-PD-1 ICI yielded an objective response rate of 15.2% (5 of 33 patients) with all (3 of 3) with lung-only metastasis responding (Cell Rep Med 2021, 2:100383). In a retrospective study that involved 14 Chinese medical centers, a partial response rate of 5% (4 of 84 patients) was reported in MSS mCRC patients administered regorafenib combined with ICIs (Cancer Immunol Immunother 2022, 71:1443-1451).

In a phase 2 study in patients from the USA with pMMR/MSS mCRC, regorafenib plus nivolumab yielded an objective response rate of 7%, with all responses observed in patients without liver metastases (E Clinical Medicine 2023, 58:101917). In this study, regorafenib was administered at 80 mg/day on a 3 weeks on/1 week off schedule and was increased to 120 mg/day if the 80 mg/day was well tolerated. Nivolumab was administered at 480 mg every 4 weeks.

Based on these data, it is possible that the regorafenib pre-treatment may have primed for responsiveness to the ICI immunotherapy in this pMMR/MSS mCRC patient that presented without liver metastases. However, in the present example, the doses and frequencies of both regorafenib and of the combination ICI immunotherapy that were administered were significantly less than the doses administered in clinical trials where objective responses were observed.

In addition, in the present example, corticosteroids were administered 6 weeks after start of ICI administration. In a retrospective single institution study, patients were evaluated in two cohorts based on timing of initiation of corticosteroids after initiation of ICI immunotherapy (≥2 months vs <2 months). The administration of corticosteroids <2 months after initiation of ICI immunotherapy was found to significantly hinder ICI efficacy (J Immunother Cancer 2021, 9).

Since regorafenib does not directly convert cold tumors to hot tumors, which is necessary for priming ICI responsiveness, and the doses and frequencies of both regorafenib and the ICI immunotherapy used in the present example were at sub-optimal therapeutic levels, combined with the early use of corticosteroids, it is unlikely that the regorafenib priming was responsible for the rare objective tumor response observed here and it is more likely that a combination that converted the cold tumors to hot was responsible.

If the restaging CT scan after AlloStim® immunotherapy reported as progressive disease by RECIST 1.1, was actually pseudoprogression due to “hot” inflammation of the tumor lesions which would make them appear to be larger than the actual tumor burden, that ICI immunotherapy would elicit a rapid tumor debulking response due to resident infiltrating effector immune cell release from suppression.

The present subject was negative for serum IL-12 at baseline. After three cycles of experimental AlloStim® immunotherapy the subject seroconverted to IL-12 positivity, supporting that the host immune system was modulated. It was previously reported that IL-12 positivity correlated with long-term survival after AlloStim® immunotherapy.

IL-12 is an effector cytokine that promotes anti-tumor immunity by activating an effector Th1 response, which is required for the activation of cytotoxic T and NK cells. IL-12 promotes production of IFN-7 which acts to upregulate PD-L1 in the tumor microenvironment, which may make these tumors more susceptible to anti-PD-L1 ICI immunotherapy.

The presence of IL-12 can have many beneficial anti-tumor effects, including: increasing production of IFN-γ from NK and T cells; stimulation of growth and cytotoxicity of activated NK cells and CD8+ and CD4+ T cells, shifting the Th1/Th2 balance in favor of the Th1 phenotype; induction of antiangiogenic cytokine and chemokine production; remodeling of the peritumoral extracellular matrix and tumor stroma, reprogramming of myeloid-derived suppressor cells, and increasing expression of MHC class I molecules necessary of cytolytic T-lymphocyte (CTL) recognition of tumor cells.

Soluble heat shock protein (HSP)-70 was also detected in the serum after AlloStim® administration. HSP-70 is a stress-inducible chaperone that is overexpressed within tumor cells, including CRC. The finding of HSP-70 in serum suggests that tumor cells have been killed in a manner where the cell membrane is disrupted (immunological cell death), releasing the HSP along with danger signals into the tumor microenvironment. HSP-70 extracellular function is believed to be immunogenic and extracellular HSP-70 can serve as an adjuvant to activate the innate immune system and can eventually lead to tumor-specific adaptive immunity. Endogenous HSP chaperone all tumor cell antigens, including self- and neo-antigens. Tumors accumulate mutations that can cause tumor-specific neo-antigen expression. Since these neo-antigens are intracellular, they may not have been previously exposed to the immune system, as the tumors sequester these neoantigens. Thus, the presence of soluble HSP-70 supports that AlloStim® modified the tumor microenvironment in a manner that caused tumor lysis and release of chaperoned neoantigens. Exposure of tumor neoantigens to the immune system increases responsiveness to ICI.

The mechanism of action of the therapeutic composition is also consistent with the conclusion that an inflammatory cold to hot conversion occurred which caused the dramatic 73% increase in target lesion size by RECIST 1.1. The immune systems of patients with metastatic cancers are dysregulated resulting in a shift toward Th2 dominance. The therapeutic composition modulates the dysregulated immune systems of these patients to a Th1 dominance using a strategy of allo-priming. “Allo-priming” refers to priming the immune system of a patient to create high titers of allo-specific Th1/CTL memory cells which can become activated. For example, the STIMVAX protocol incorporated a first series of intradermal injections of the therapeutic composition. The host rejection of the intentionally mis-matched cells shortly after administration results in increased titers of allo-specific Th1 and CTL cells, modulating the resident Th1/Th2 balance.

These allo-specific cells elicited after intradermal injections are non-specifically activated by cytokine release after intravenous infusion of the therapeutic composition through a bystander activation mechanism. Activated T-cells can extravasate and local inflammation attracts these cells into tissues and sites of inflammation, including tumors. Thus, the intravenous infusion of the therapeutic composition after allo-priming can convert “cold” tumors into “hot” tumors with extensive infiltration of Th1/CTL memory cells, which could possibly account for the 73% increase in target lesion size.

Inflamed tumor lesions can enlarge and appear as progressive disease by RECIST 1.1. However, the putative anti-tumor mechanism leading to tumor debulking immunity may take several additional months before a radiological response can be detected and patients are often removed from the treatment protocols before a later assessment can be conducted.

It was hypothesized that based on the mechanism of action of AlloStim® that radiological progression after 3 cycles likely represents a beneficial immune response that has primed the tumor lesions for an eventual debulking anti-tumor response. To support this hypothesis, a short course of combination ICI immunotherapy was administered. Since pMMR/MSS mCRC is known not to be responsive to ICI immunotherapy, it was predicted that if a rapid tumor debulking response was observed, this would provide evidence supporting that the tumor lesions had been previously primed with infiltrating effector immune cells.

### CONCLUSION

The available evidence makes it appear likely that AlloStim® played a role in eliciting the objective response observed after regorafenib and combination ICI immunotherapy.

### FIG. 1: CT Scan

Longitudinal changes in two lung target lesions (arrows). The slices are adjusted to show the view with the measurement in the longest tumor diameter. Circles indicate presence or absence of non-target lesions in the selected slices. An increase in size of non-target existing lesions and appearance of new non-target lesions seen on post-AlloStim Scan B, compared to Scan A, baseline. Elimination or reduction in size of the non-target lesions seen in the post-ICI Scan C. According to RECIST 1.1, the Scan B, compared to Scan A, is scored as progressive disease. The Scan C, compared to Scan B, is scored as partial response. The Scan C, compared to Scan A, is scored as stable disease.

### FIG. 2: IL-12 and HSP70 Serum Levels During Allostim Administration

Whole blood samples were collected longitudinally from subjects in SST Tiger Top tubes. The tubes were spun at 3000 rpm and shipped overnight at 2-80 C to the central lab facility where the serum was aseptically transferred to cryotubes and stored at −800 C until analysis. For analysis, samples were diluted 1:2 and plated in triplicate on ELISA plates (R&D Systems) and incubated for 2-3 h at RT. The plates were read on a Cytation 7 plate reader (Agilent BioTek) at 650 nm absorbance. A standard curve was generated using known samples. Quantitative levels were determined by comparing absorbance values to the standard curve. The bar graph shows the mean+/−SE at each sample time point.

FIG. 5: Matched CT Scans and Biopsies of Liver Target Lesions in Two MSS MCRC Subjects at Baseline and at Day 84 after Allostim®

Subject #1 shows extensive tumor (red circle) on the periphery of areas of fibrosis without immune cell infiltration. The corresponding CT scan shows the target tumor. After 3 cycles of AlloStim®, the re-staging CT scan shows progressive disease. However, the corresponding biopsy shows areas of coagulative necrosis and immune cell infiltration. Subject #2 has almost completely solid tumor at baseline. The re-staging CT scan indicates progressive disease, however, note the extensive peri-tumoral inflammation. The corresponding biopsy indicates large area of tumor necrosis and tumor admixed with immune cells.

Although the present disclosure has been described with reference to preferred embodiments, workers skilled in the art will recognize that changes may be made in form and detail without departing from the spirit and scope of the disclosure.

What is claimed is:

1. A method of treating metastatic colorectal cancer in a patient, comprising:
administering activated allogeneic CD4+ T-cells to a patient; and
administering at least one immune checkpoint inhibitor to the patient.

2. The method of claim 1 wherein the immune checkpoint inhibitor comprises any one of ipilimumab; pembrolizumab; nivolumab; atezolizumab; avelumab; durvalumab; cemiplimab; tremelimumab; retifanlimab; dostarlimab; toripalimab; or combinations thereof.

3. The method of claim 1 wherein the immune checkpoint inhibitor targets CTLA-4, PD-1, PD-L1, and/or LAG-3 checkpoint molecules.

4. The method of claim 1 further comprising administering regorafenib.

5. The method of claim 1 wherein the patient has a tumor.

6. The method of claim 5 further comprising reducing the size of the tumor or eliminating the tumor.

7. The method of claim 1 wherein the activated allogeneic CD4+ T-cells comprises an activated Th1 memory phenotype: CD4+, CD45RO+, CD62Llo, CD40Lhi, CD25+, IFN-γ+, and IL-4−.

8. The method of claim 7 wherein the activated allogeneic CD4+ T-cells are maintained in an activated state by continuous attachment to CD3/CD28-monoclonal antibody-coated microparticles.

9. The method of claim 1 wherein administration is intradermal or intravenous and is administered more than once.

10. The method of claim 1 wherein the CD4+ T-cells are differentiated and expanded ex-vivo to become Th1-like effector cells prior to administration.

11. The method of claim 10 wherein the Th1-like effector cells are stored in liquid nitrogen as an intermediate prior to administration.

12. The method of claim 11 wherein the Th1-like effector cells after storage in the liquid nitrogen are thawed and activated with microbeads causing differentiation of the cells to an activated Th1 memory cell phenotype prior to administration.

13. A method of treating metastatic colorectal cancer in a patient, comprising:
priming a patient comprising:
increasing a titer of circulating CD4+ Th1 memory cells in the patient by administering a therapeutic composition to the patient, wherein the patient has a tumor and the therapeutic composition comprises activated allogeneic CD4+ T-cells; and
activating the circulating CD4+ Th1 memory cells in the patient; and
administering an immune checkpoint inhibitor to the patient.

14. The method of claim 13 wherein the tumor comprises a “cold” tumor characterized by the tumor lacking tumor-infiltrating lymphocytes.

15. The method of claim 14 wherein the priming converts the “cold” tumor to a “hot” tumor, wherein the “hot” tumor comprises one or more of an increased level of tumor-infiltrating lymphocytes, an interferon-T signature, and/or an upregulated PD-L1 expression.

16. A composition for treating metastatic colorectal cancer in a patient, comprising:
activated allogeneic CD4+ T-cells; and
an immune checkpoint inhibitor.

17. The composition of claim 16 wherein the immune checkpoint inhibitor comprises any one of ipilimumab; pembrolizumab; nivolumab; atezolizumab; avelumab; durvalumab; cemiplimab; tremelimumab; retifanlimab; dostarlimab; toripalimab; or combinations thereof.

18. The composition of claim 16 wherein the immune checkpoint inhibitor targets CTLA-4, PD-1, PD-L1, and/or LAG-3 checkpoint molecules.

19. The composition of claim 16 further comprising a multi-kinase inhibitor.
